(19) United States
(12) Patent Application Publication
(10) Pub. No.: US 2023/0267847 A1
(43) Pub. Date: Aug. 24, 2023

(54) VIRTUAL ASSESSMENT INSTRUMENTS FOR ANXIETY AND SELF-ESTEEM

(71) Applicant: Mohammed Naji Aldossari, Rochester Hills, MI (US)

(72) Inventor: Mohammed Naji Aldossari, Rochester Hills, MI (US)

(21) Appl. No.: 17/652,370

(22) Filed: Feb. 24, 2022

(51) Int. Cl. G09B 5/06; G06F 3/01; G16H 20/70

(52) U.S. Cl. CPC G09B 5/06; G06F 3/011; G16H 20/70; A63F 2003/0489

(57) ABSTRACT

A system and method for providing anxiety and/or self-esteem assessments of a user, such as a mental health counselor in training, a student or a client, using a virtual reality device. The method includes programming the virtual reality device with a plurality of scenarios for counseling a virtual patient, selecting various parameters by the user to identify one of the scenarios, operating the virtual reality device to initiate the selected scenario and perform a virtual counseling session with the virtual patient, and assessing the anxiety and self-esteem of the user from the virtual counseling session. The method could include uploading the virtual counseling session and assessments to a virtual reality server platform to be processed and analyzed.

### BACKGROUND

### Field

This disclosure relates generally to providing anxiety and self-esteem assessments for a user and, more particularly, to a system and method for providing anxiety and self-esteem assessments for a user, such as a mental health counselor in training, a student or a client, using a virtual reality device.

### Discussion of the Related Art

Students go through rigorous training to become licensed mental health counselors. That training includes classroom learning from an instructor, watching counseling training videos, roll playing between classmates, etc. This training also includes active clinical counseling with real patients. Mental health counseling training by students can be very stressful because of the anxiety that comes with this type of training as a result of many factors, such as fear of being judged, fear of poor performance, fear of being criticized, etc. Add to that there are often cultural differences between the counselor and the patient, such as race, religion, sexual orientation, gender, etc., that cause the anxiety level to increase and the confidence level of the student to be reduced.

Virtual reality (VR) is a computer-generated simulation of a three-dimensional image or environment that can be interacted with in a seemingly real or physical way by a person using special electronic equipment, such as a helmet with a screen. A VR simulation can be similar to or completely different from the real world. Applications of virtual reality include entertainment, such as video games, education, such as medical or military training, and business, such as virtual meetings. It may be possible to provide a system and method where training for mental health counselors can be performed virtually to reduce anxiety and increase confidence.

### SUMMARY

The following discussion discloses and describes a system and method for providing anxiety and/or self-esteem assessments of a user, such as a mental health counselor in training, a student or a client, using a virtual reality device. The method includes programming the virtual reality device with a plurality of scenarios for counseling a virtual patient, selecting various parameters by the user to identify one of the scenarios, operating the virtual reality device to initiate the selected scenario and perform a virtual counseling session with the virtual patient, and assessing the anxiety and/or self-esteem of the user from the virtual counseling session. The method could include uploading the virtual counseling session and assessments to a virtual reality server platform to be processed and analyzed.

Additional features of the disclosure will become apparent from the following description and appended claims, taken in conjunction with the accompanying drawings.

### BRIEF DESCRIPTION OF THE DRAWINGS

FIG. 1 is a flow chart diagram showing a system and method for using a virtual reality device for providing mental health counseling skills training for a counselor in training;

FIG. 2 is an illustration of a screen in the virtual reality device showing a dashboard for selecting options and scenarios; and

FIG. 3 is a flow chart diagram showing a system and method for using a virtual reality device for providing anxiety and self-esteem assessments for a user.

### DETAILED DESCRIPTION OF THE EMBODIMENTS

The following discussion of the embodiments of the disclosure directed to a system and method for providing anxiety and/or self-esteem assessments for a user using a virtual reality device is merely exemplary in nature, and is in no way intended to limit the disclosure or its applications or uses.

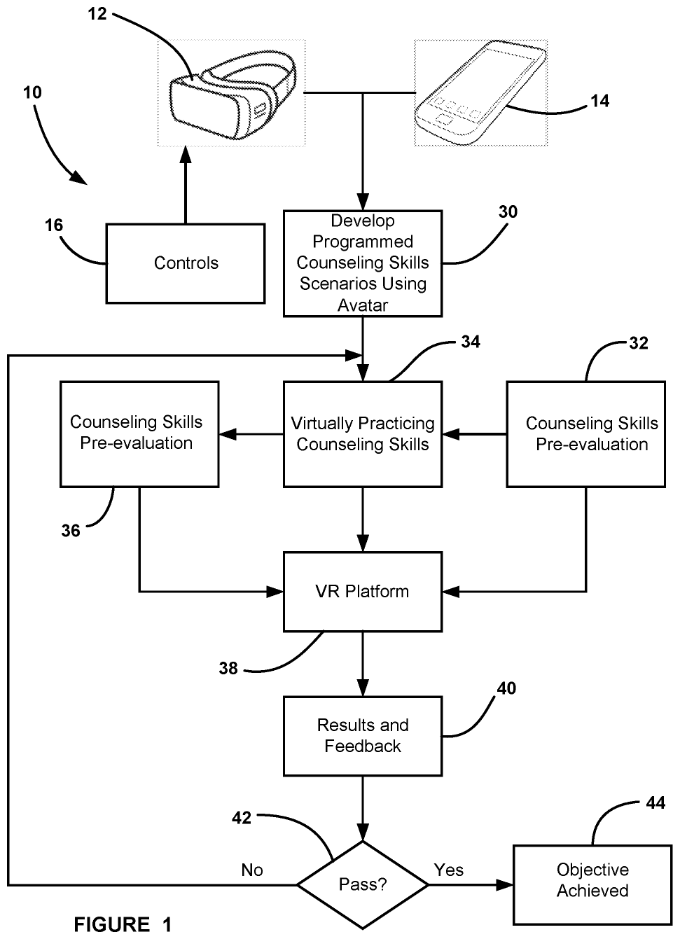

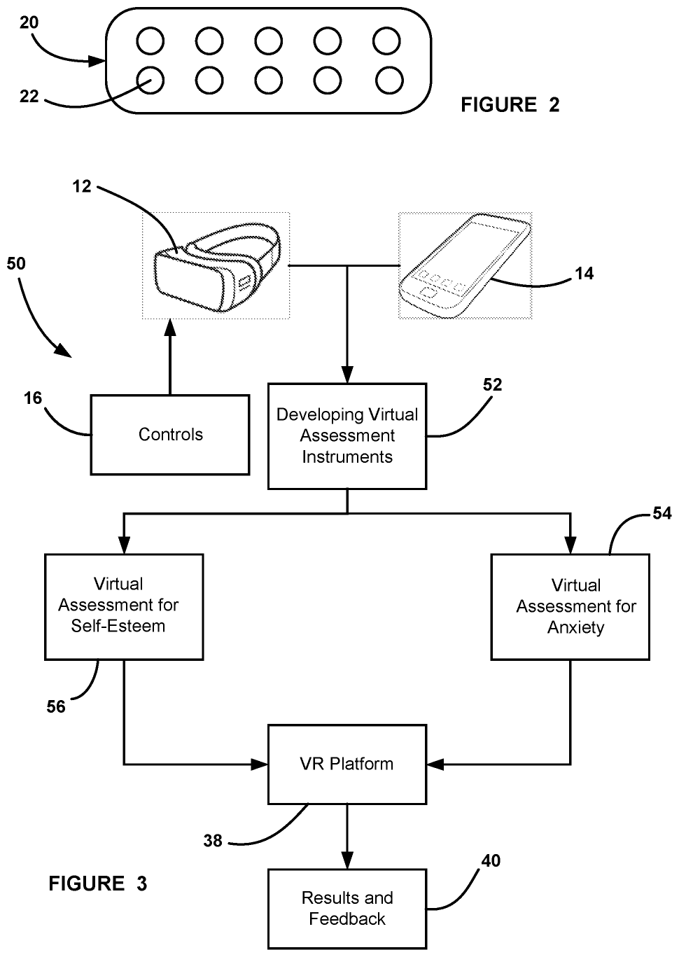

FIG. 1 is a flow chart diagram 10 showing a system and method for using a virtual reality device, for example, a virtual reality headset 12 or a smart phone 14 having virtual reality capabilities, for providing skills training for mental health counseling. The headset 12 and the smart phone 14 will include all of the necessary memories, screens, programs, Wi-Fi radios, processors, programs, etc. to perform the virtual reality animations consistent with the discussion herein. A counselor in training will put on the headset 12 and use virtual reality controls 16 coupled to the headset 12 by, for example, a Wi-Fi protocol to open a virtual training dashboard 20 having a number of selection icons 22 with drop down menus, see FIG. 2, that allow the counselor in training to select various input parameters and selection criteria to perform a certain simulated counseling training scenario consistent with the discussion herein. The icons 22 allow the counselor in training to develop a limitless number of virtual counseling scenarios using an Avatar as a virtual patient or otherwise. The scenarios may be developed by selecting the type of counseling, counseling skill level, counseling subject matter, counseling location, i.e., session location, counseling time, patient age, patient race, patient sex, patient education level, patient sexual orientation, patient religion, etc. at box 30.

Prior to the counselor in training initiating the selected virtual reality counseling session, the counselor in training will perform a counseling skills pre-evaluation process at box 32. This pre-evaluation process could come in a number of forms based on how to respond to certain situations. Once the scenario has been developed and the pre-evaluation has been performed, the counselor in training will begin the virtual reality counseling session at box 34, which will be a virtual reality interactive session based on the programmed scenario as selected by the counselor in training. This programming will allow the counselor in training to engage the selected animation patient, where the animation patient tells a pre-programmed story and the counselor in training will ask questions and the responses from the animation patient will be pre-programmed. Once the virtual reality training session is complete, then the counselor in training will go through a counseling skills post-evaluation process at box 36, which could come in a number of forms based on how to respond to certain situations.

The pre-evaluation information, the post-evaluation information and the actual training session is uploaded to a VR server platform 38 that stores and processes all of the data and allows it to be automatically evaluated by a suitable analysis program and viewed, for example, by the counselor in training or an instructor. The platform 38 is accessed by, for example, logging on to a computer connected to the internet, where results and feedback can be determined at box 40. A pass or fail determination can be made at decision diamond 42 from the results and feedback, where objectives are achieved at box 44 for a pass and a new session can be initiated at the box 34 for a fail. By providing mental health skills training in this manner, students can confidently develop their skills in a safe, comfortable and effective manner.

In another embodiment, the headset 12 or the smart phone 14 is programmed with the information necessary for the virtual reality environment to assess the anxiety and self-esteem, i.e., confidence, of a user, such as a mental health counselor in training, a student or a client, during a virtual reality simulated counseling session. This embodiment is illustrated in FIG. 3, which is a flow chart diagram 50 of a system and method for the user in training anxiety and self-esteem during a simulated counseling session. One or more simulated counseling exercises are developed at box 52 in the manner discussed above. Specifically, the user will put on the headset 12 and use the virtual reality controls 16 to open the virtual training dashboard 20 and allow the user to select various input parameters and selection criteria using the icons 22 to perform a certain simulated counseling training scenario.

For the anxiety assessment, the user will perform the virtual reality counseling session at box 54, which will be a virtual reality interactive session based on the programmed scenario as selected by the user. The virtual reality programming will automatically assess the anxiety of the user during the simulated training session using, for example, a questionnaire. This anxiety assessment is performed before the virtual counseling session and after the virtual counseling session.

For the self-esteem assessment, the user will perform the virtual reality counseling session at box 56, which will be a virtual reality interactive session based on the programmed scenario as selected by the user. The virtual reality programming will automatically assess the self-esteem of the user during the simulated training session using, for example, a questionnaire with predetermined relevant questions. This self-esteem assessment is performed before the virtual counseling session and after the virtual counseling session.

The information and data from the virtual counseling session and the anxiety and self-esteem assessments are uploaded to the VR platform 38 where it can be evaluated and analyzed and feedback can be provided at the box 40 in the manner discussed above.

The foregoing discussion discloses and describes merely exemplary embodiments of the present disclosure. One skilled in the art will readily recognize from such discussion and from the accompanying drawings and claims that various changes, modifications and variations can be made therein without departing from the spirit and scope of the disclosure as defined in the following claims.

What is claimed is:

1. A method for assessing a user, said method comprising:
programming a virtual reality device with a plurality of scenarios for counseling a virtual patient;
selecting various parameters by the user to identify one of the scenarios;
operating the virtual reality device to initiate the selected scenario and perform a virtual counseling session with the virtual patient; and
assessing the anxiety of the user from the virtual counseling session.

2. The method according to claim 1 wherein assessing the anxiety of the user includes using a questionnaire.

3. The method according to claim 1 wherein assessing the anxiety of the user includes assessing the user before the virtual counseling session and after the virtual counseling session.

4. The method according to claim 1 further comprising uploading the virtual counseling session and the assessment to a virtual reality server platform to be processed and analyzed.

5. The method according to claim 4 further comprising providing feedback for the analyzed virtual counseling session.

6. The method according to claim 1 wherein selecting various parameters by the user includes selecting one or more of type of counseling, counseling skill level, counseling subject matter, counseling location, counseling time, patient age, patient race, patient sex, patient education level, patient sexual orientation and patient religion.

7. The method according to claim 1 wherein the virtual reality device is a headset or a smart phone.

8. The method according to claim 1 wherein the user is a mental health counselor in training, a student or a client.

9. A method for assessing a user, said method comprising:
programming a virtual reality device with a plurality of scenarios for counseling a virtual patient;
selecting various parameters by the user to identify one of the scenarios;
operating the virtual reality device to initiate the selected scenario and perform a virtual counseling session with the virtual patient; and
assessing the self-esteem of the user from the virtual counseling session.

10. The method according to claim 9 wherein assessing the self-esteem of the user includes using a questionnaire.

11. The method according to claim 9 wherein assessing the self-esteem of the user includes assessing the user before the virtual counseling session and after the virtual counseling session.

12. The method according to claim 9 further comprising uploading the virtual counseling session and the assessment to a virtual reality server platform to be processed and analyzed.

13. The method according to claim 12 further comprising providing feedback for the analyzed virtual counseling session.

14. The method according to claim 9 wherein selecting various parameters by the user includes selecting one or more of type of counseling, counseling skill level, counseling subject matter, counseling location, counseling time, patient age, patient race, patient sex, patient education level, patient sexual orientation and patient religion.

15. The method according to claim 9 wherein the virtual reality device is a headset or a smart phone.

16. The method according to claim 9 wherein the user is a mental health counselor in training, a student or a client.

17. A method for assessing a user, said method comprising:
programming a virtual reality device with a plurality of scenarios for counseling a virtual patient;
selecting various parameters by the user to identify one of the scenarios;
operating the virtual reality device to initiate the selected scenario and perform a virtual counseling session with the virtual patient;
assessing the self-esteem of the user from the virtual counseling session; and
assessing the anxiety of the user from the virtual counseling session.

18. The method according to claim 17 wherein assessing the self-esteem of the user includes using a questionnaire.

19. The method according to claim 17 wherein assessing the self-esteem of the user includes assessing the user before the virtual counseling session and after the virtual counseling session.

20. The method according to claim 17 wherein the user is a mental health counselor in training, a student or a client.
